(19) United States
(12) Patent Application Publication
(10) Pub. No.: US 2025/0336469 A1
(43) Pub. Date: Oct. 30, 2025

(54) AUTOMATED KINETIC MODEL GENERATION FOR BIOCHEMICAL PATHWAYS

(71) Applicant: X Development LLC, Mountain View, CA (US)

(72) Inventors: Jeffrey David Orth, Alameda, CA (US); Joseph Mark Dale, Sunnyvale, CA (US); John Ata Bachman, Newton, MA (US)

(21) Appl. No.: 19/170,980

(22) Filed: Apr. 4, 2025

Related U.S. Application Data
(60) Provisional application No. 63/639,980, filed on Apr. 29, 2024.

(51) Int. Cl. G16B 5/00; G06F 30/27; G16B 40/20

(52) U.S. Cl. CPC G16B 5/00; G06F 30/27; G16B 40/20

(57) ABSTRACT

Methods, systems, and apparatus, including computer programs encoded on a computer storage medium, for training kinetic models on experimental data of biochemical pathways. In one aspect, a method comprises: receiving chemical reaction data for a biochemical pathway; automatically generating data defining a kinetic model of the biochemical pathway based on the chemical reaction data; obtaining experimental data for the biochemical pathway; training the kinetic model on the experimental data using a numerical optimization technique to optimize an objective function that measures a discrepancy between: (i) simulated data characterizing the biochemical pathway that is generated using the kinetic model, and (ii) the experimental data characterizing the biochemical pathway; and outputting the kinetic model of the biochemical pathway after training the set of kinetic model parameters.

### CROSS-REFERENCE TO RELATED APPLICATION

This specification claims priority to U.S. Provisional Application No. 63/639,980, filed on Apr. 29, 2024. The disclosure of the prior application is considered part of and is incorporated by reference in the disclosure of this application.

### BACKGROUND

This specification relates to generating models of biochemical pathways.

A biochemical pathway includes a set of linked chemical reactions involved in the metabolism of an organism. The chemical reactions within a biochemical pathway can be mediated by catalyzing and inhibiting compounds for the reactions.

Biochemical pathways can be used to synthesize chemical compounds, such as pharmaceuticals, biofuels, industrial enzymes, and so on.

### SUMMARY

This specification describes a system implemented as computer programs on one or more computers in one or more locations that can automatically generate a kinetic model for simulating a biochemical pathway. The system can use the generated kinetic model to optimize the production rate of an output compound of the biochemical pathway.

According to a first aspect, there is provided a method performed by one or more computers, the method comprising: receiving data characterizing a plurality of chemical reactions included in a biochemical pathway; processing the data characterizing the plurality of chemical reactions included in the biochemical pathway to automatically generate data defining a kinetic model of the biochemical pathway, wherein the kinetic model comprises a set of kinetic model parameters; obtaining experimental data characterizing the biochemical pathway, including one or both of: metabolite concentration data measuring concentrations of one or more metabolites included in one or more chemical reactions in the biochemical pathway, and reaction flux data for one or more chemical reactions included in the biochemical pathway; training the set of kinetic model parameters of the kinetic model on the experimental data characterizing the biochemical pathway using a numerical optimization technique to optimize an objective function that measures a discrepancy between: (i) simulated data characterizing the biochemical pathway that is generated using the kinetic model, and (ii) the experimental data characterizing the biochemical pathway; and outputting the kinetic model of the biochemical pathway after training the set of kinetic model parameters.

In some implementations, obtaining the experimental data characterizing the biochemical pathway comprises: obtaining respective experimental data characterizing the biochemical pathway under each of a plurality of respective experimental conditions.

In some implementations, the set of kinetic model parameters comprises: (i) one or more kinetic model parameters identified as global kinetic model parameters that are invariant across experimental conditions, and (ii) one or more kinetic model parameters that are identified as local kinetic model parameters that vary across experimental conditions; and training the set of kinetic model parameters on the experimental data characterizing the biochemical pathway comprises: determining a respective value of each global kinetic model parameter by training the global kinetic model parameters on experimental data corresponding to each of the plurality of experimental conditions; and determining, for each experimental condition of the plurality of experimental conditions, a respective value of each local kinetic model parameter that is specific to the experimental condition by training the local kinetic model parameters only on experimental data corresponding to the experimental condition.

In some implementations, the global kinetic model parameters comprise enzymatic parameters including one or more of: one or more enzyme turnover rates (k_{cat}), one or more dissociation constants (K_{d}), or one or more inhibition constants (Kᵢ).

In some implementations, the local kinetic model parameters comprise one or more boundary metabolite concentrations.

In some implementations, training the set of kinetic model parameters of the kinetic model on the experimental data characterizing the biochemical pathway comprises: performing the training of the set of kinetic model parameters a plurality of times, each time with a different random initialization of values of the set of kinetic model parameters, to generate an ensemble of trained values of the set of kinetic model parameters.

In some implementations, processing the data characterizing the plurality of chemical reactions included in the biochemical pathway to automatically generate data defining a kinetic model of the biochemical pathway comprises: automatically identifying a respective reaction rate expression for each of the plurality of chemical reactions, wherein each reaction rate expression is parametrized by one or more respective kinetic model parameters of the kinetic model; and processing the reaction rate expressions for the plurality of chemical reactions to generate the data defining the kinetic model of the biochemical pathway.

In some implementations, the biochemical pathway comprises a plurality of metabolites, wherein each metabolite is included in one or more chemical reactions in the biochemical pathway as a reactant or as a product; and processing the reaction rate expressions for the plurality of chemical reactions to generate the data defining the kinetic model of the biochemical pathway comprises, for each of one or more metabolites included in the biochemical pathway: generating a model of a rate of change of a concentration of the metabolite with respect to time as a combination of the reaction rate expressions for each chemical reaction that includes the metabolite in the biochemical pathway.

In some implementations, for one or more of the plurality of chemical reactions, automatically identifying a respective reaction rate expression for the chemical reaction comprises: automatically identifying the reaction rate expression for the chemical reaction based on one or more of: a number of reactants in the chemical reaction; a number of products of the chemical reaction; or an enzymatic reaction mechanism of the chemical reaction.

In some implementations, processing the data characterizing the plurality of chemical reactions included in the biochemical pathway to automatically generate data defining the kinetic model comprises: identifying, as a boundary metabolite, each metabolite in the biochemical pathway that is: included in only one chemical reaction in the biochemical pathway, or is included only as a reactant or only as a product of an irreversible chemical reaction in the biochemical pathway, or both; and modifying the kinetic model to set, for each metabolite identified as a boundary metabolite, a concentration of the metabolite to be a constant instead of a variable value.

In some implementations, processing the data characterizing the plurality of chemical reactions included in the biochemical pathway to automatically generate data defining the kinetic model comprises: identifying, as an extrinsically-connected metabolite, each metabolite in the biochemical pathway that is included in one or more chemical reactions outside the biochemical pathway in a genome-scale model of metabolism; and modifying the kinetic model to include, for each extrinsically-connected metabolite, a respective drain chemical reaction that consumes the extrinsically-connected metabolite.

In some implementations, the method further comprises: determining, for each of one or more drain chemical reactions, a respective expected flux of the drain chemical reaction using the genome-scale model of metabolism; and wherein, for each of one or more drain chemical reactions, the objective function used for training the set of kinetic model parameters of the kinetic model further measures a discrepancy between: (i) a simulated flux of the drain chemical reaction that is generated using the kinetic model, and (ii) the expected flux of the drain chemical reaction.

In some implementations, determining, for each of one or more drain chemical reactions, the respective expected flux of the drain chemical reaction comprises: obtaining experimental data characterizing respective uptake or production rates of one or more metabolites; determining, based on the experimental data characterizing the respective uptake or production rates of the one or more metabolites and using a numerical optimization, a respective flux of each chemical reaction in the genome-scale model of metabolism; and determining, for each drain chemical reaction associated with a metabolite, the respective expected flux as a combination of fluxes of chemical reactions in the genome-scale model of metabolism that: (i) produce or consume the metabolite, and (ii) are not included in the biochemical pathway.

In some implementations, the set of kinetic model parameters of the kinetic model of the biochemical pathway comprise one or more of: one or more equilibrium constants (K_{eq}); or one or more enzyme turnover rates (k_{cat}); or one or more dissociation constants (K_{d}); or one or more inhibition constants (Kᵢ); or one or more drain reaction constants (K_{drain}); or one or more enzyme concentrations; or one or more boundary metabolite concentrations.

In some implementations, processing the data characterizing the plurality of chemical reactions included in the biochemical pathway to automatically generate data defining the kinetic model of the biochemical pathway comprises, for one or more kinetic model parameters of the kinetic model: automatically retrieving data specifying a respective initial value of the kinetic model parameter from one or more databases of chemical reaction data.

In some implementations, processing the data characterizing the plurality of chemical reactions included in the biochemical pathway to automatically generate data defining the kinetic model of the biochemical pathway comprises: obtaining one or more Michaelis-Menten constants (Kₘ) associated with an enzyme; determining one or more dissociation constants (K_{d}) for the enzyme from the one or more Michaelis-Menten constants (Kₘ) associated with the enzyme, comprising: performing a numerical optimization to determine optimized values of the one or more dissociation constants (K_{d}) that minimize an error between: (i) predicted chemical reaction flux values generated using a Michaelis-Menten equation parametrized by the one or more Michaelis-Menten constants (Kₘ), and (ii) predicted chemical reaction flux values generated using a kinetic model parametrized by the one or more dissociation constants (K_{d}); and after optimizing values of the one or more dissociation constants (K_{d}), including the one or more dissociation constants (K_{d}) in the set of kinetic model parameters.

In some implementations, processing the data characterizing the plurality of chemical reactions included in the biochemical pathway to automatically generate data defining the kinetic model of the biochemical pathway comprises: processing the data characterizing the plurality of chemical reactions to identify one or more chemical reactions with incomplete chemical reaction data; and automatically completing the chemical reaction data for each chemical reaction that is identified as having incomplete chemical reaction data, comprising, for each chemical reaction that is identified as having incomplete chemical reaction data: automatically identifying one or more features that are not included in the received data characterizing the chemical reaction; and automatically retrieving data specifying the one or more features that are not included in the received data characterizing the chemical reaction from one or more databases of chemical reaction data.

In some implementations, for one or more of the chemical reactions that are identified as having incomplete reaction data, automatically retrieving data specifying the one or more features that are not included in the received data characterizing the chemical reaction from the database of chemical reaction data comprises automatically retrieving data specifying one or more of: a stoichiometry of the chemical reaction; or one or more catalyzing enzymes for the chemical reaction; or one or more inhibitor metabolites for the chemical reaction; or an enzymatic reaction mechanism for the chemical reaction.

In some implementations, outputting the kinetic model of the biochemical pathway further comprises: determining, using the kinetic model of the biochemical pathway, that changing a respective concentration of each of one or more target enzymes is predicted to increase a rate of production of an output of the biochemical pathway.

In some implementations, determining, using the kinetic model of the biochemical pathway, that changing a respective concentration of each of one or more target enzymes is predicted to increase a rate of production of an output of the biochemical pathway comprises: performing a numerical optimization of an objective function that measures a production rate of an output produced by the biochemical pathway over a space of possible values of enzyme concentration parameters included in the set of kinetic model parameters of the kinetic model; and identifying the one or more target enzymes based on a result of the numerical optimization of the objective function that measures the production rate of the output produced by the biochemical pathway.

In some implementations, the output of the biochemical pathway comprises a pharmaceutical or a biofuel or an industrial enzyme.

In some implementations, the method further comprises determining that a genome of a microorganism should be modified to increase expression of the one or more target enzymes.

In some implementations, the method further comprises genetically modifying the microorganism to increase the expression of the one or more target enzymes.

In some implementations, the method further comprises cultivating a population of the genetically modified microorganisms.

According to another aspect there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect, there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

Accurately modeling biochemical pathways is a significant computational challenge. For example, a metabolic network may include thousands of individual chemical reactions, with each reaction consuming particular reactants, producing particular products, and being mediated by particular catalysts and inhibitors (e.g., enzymes, metabolites, etc.). The chemical reactions of a biochemical pathway are intricately coupled, with each reaction consuming products of other reactions, producing reactants for other reactions, and potentially sharing mediating enzymes with other reactions in the pathway. Modeling biochemical pathways to describe how the pathways proceed as observed within organisms (e.g., as part of metabolic networks) is therefore challenging.

Biochemical pathways can be optimized to maximize production rates of the synthesized chemical compounds. In particular, a biochemical pathway can be optimized by genetically modifying a microorganism to perform the biochemical pathway using optimized enzyme concentrations (e.g., as optimized to increase catalyst concentrations, decrease inhibitor concentrations, etc.). Optimizing biochemical pathways to increase the production of target outputs requires accurately predicting how the pathways would proceed for a variety of enzyme concentrations (e.g., including enzyme concentrations that have not yet been observed within organisms), which can be a more challenging task compared to modeling the biochemical pathways to describe how the pathways have been observed proceeding within organisms.

The described systems can obtain data specifying biochemical pathways and automatically generate kinetic models for the biochemical pathways. In particular, the described systems can automatically generate kinetic models corresponding to the biochemical pathways using systems of coupled differential equations that accurately model the individual chemical reactions within the biochemical pathways. The described systems can then use the generated kinetic models to optimize the production rate of target products within the biochemical pathways. For example, the described systems may optimize a biochemical pathway to determine that increasing the expression of particular enzymes will increase a predicted production rate of a target output of the pathway.

Conventional methods for modeling biochemical pathways are often dedicated to accurately modeling the pathways as the pathways have been observed within organisms. For example, conventional genome scale models of metabolism can model how a complete metabolic network of a microorganism (e.g., by including data characterizing enzyme properties, equilibrium properties of reactions within the network, etc.) proceeds within the microorganism. However, conventional methods are less suited to efficiently and accurately optimizing the biochemical pathways in order to increase production of individual products.

The described systems can accurately model subsets of reactions for biochemical pathways that relate to the production of the target outputs. The described systems can generate kinetic models for the biochemical pathways that more accurately model dynamics of reactions within the pathways (e.g., by modeling the effects of gene expression, regulation mechanisms, etc.). In particular, the described systems can generate a kinetic model for a subset of reactions from a biochemical pathway by determining and applying certain boundary conditions to the concentrations of the reactants and products for the subset, which enables the described systems to model the subset of reactions as a part of the biochemical pathway without generating a kinetic model of the complete biochemical pathway. The described systems can therefore optimize biochemical pathways with less computational cost (e.g., in terms of computational time, power consumption, etc.) than conventional methods.

Altering and optimizing biochemical pathways enables an efficient synthesis of chemical products (e.g., pharmaceuticals, biofuels, industrial enzymes, etc.). In particular, microorganisms can be genetically modified to express an altered and optimized biochemical pathway. In some implementations, the described systems can produce instructions for genetically modifying microorganisms (e.g., by indicating particular gene sequences to add or remove) and for cultivating populations of genetically modified microorganisms in order to produce the target outputs. By automatically modeling and optimizing the biochemical pathways to increase the production rates of target outputs, the described systems can therefore enable more efficient chemical synthesis of a variety of chemical products.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example model generation system.

FIG. 2 illustrates an example biochemical pathway.

FIG. 3A illustrates using a kinetic model to generate the simulated reaction data for a biochemical pathway.

FIG. 3B illustrates a biochemical pathway as simulated by a kinetic model.

FIG. 4A illustrates an example chemical reaction as simulated by a kinetic model.

FIG. 4B illustrates an example drain reaction as modeled by a kinetic model.

FIG. 4C illustrates an example source reaction as modeled by a kinetic model.

FIG. 5 is a flow diagram of an example process for generating and training a kinetic model for a biochemical pathway.

FIG. 6 is a flow diagram of an example process for generating a kinetic model for a biochemical pathway.

FIG. 7 is a flow diagram of an example process for pre-training a kinetic model using experimental data that characterizes modeled chemical reactions in isolation.

FIG. 8 is a flow diagram of an example process for training a kinetic model of a biochemical pathway.

FIG. 9 illustrates training a kinetic model using experimental data from different experimental conditions.

FIG. 10 is a flow diagram of an example process for optimizing production of a target compound using a kinetic model of a biochemical pathway.

Like reference numbers and designations in the various drawings indicate like elements.

### DETAILED DESCRIPTION

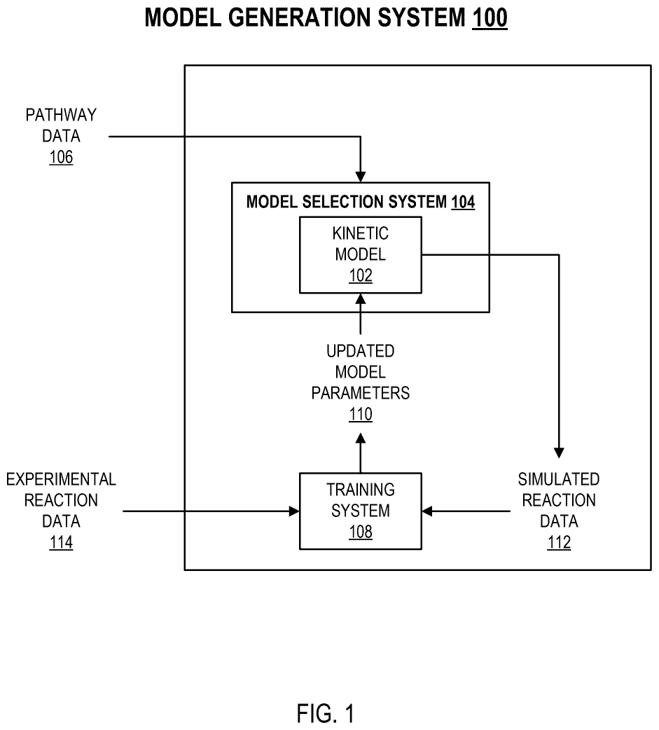

FIG. 1 shows an example model generation system 100. The model generation system 100 is an example of a system implemented as computer programs on one or more computers in one or more locations in which the systems, components, and techniques described below are implemented.

The model generation system 100 can generate and train a kinetic model 102 of a biochemical pathway.

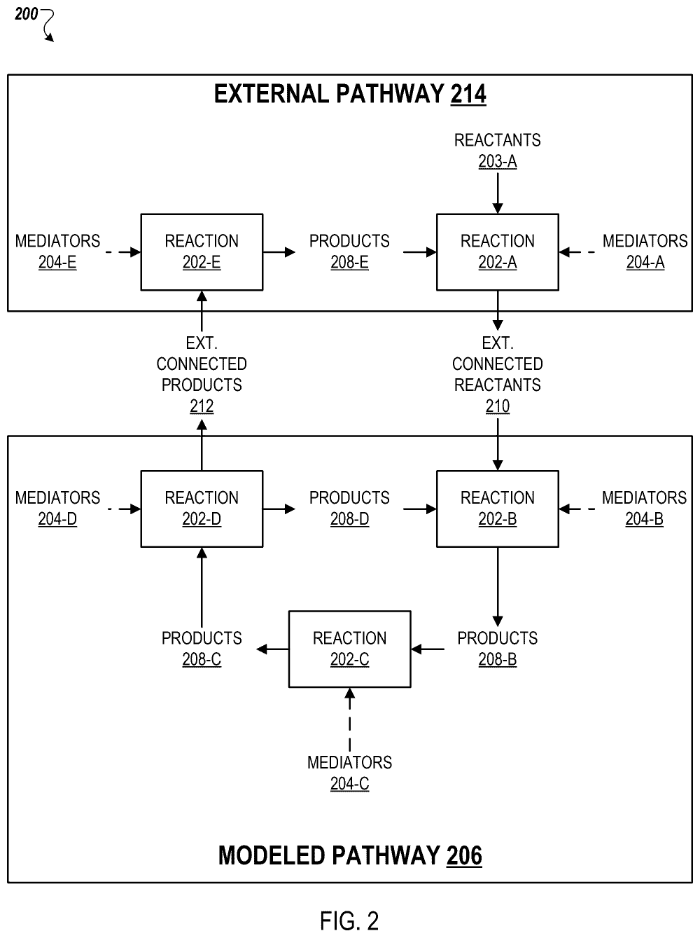

The biochemical pathway is a network of linked chemical reactions for a biological process. Each chemical reaction within the biochemical pathway consumes certain reactants and generates certain products. In particular, the chemical reactions within the pathway can be linked by their reactants and products (e.g., a reaction that generates a given compound as a product can be linked within the pathway to another reaction that consumes the given compound as a reactant). The biochemical pathway can, as a whole, generate chemical products such as biofuels, pharmaceuticals, and so on (e.g., as products of chemical reactions within the pathway) by consuming input chemical reactants (e.g., as reactants to chemical reactions within the pathway). An example biochemical pathway is described in more detail below with respect to FIG. 2.

Each chemical reaction within the biochemical pathway proceeds at a reaction rate (e.g., a rate at which the reaction creates the products and consumes the reactants) that determines how the concentrations of the reaction's products and reactants change over time. The reaction rate for a chemical reaction depends on various physical conditions surrounding the chemical reaction. For example, the reaction rate can depend on concentrations of the reactants and products (e.g., increasing the concentrations of the reactants can increase the rate of the reaction and increasing the concentrations of the products can decrease the rate of the reaction). As another example, the reaction rate can depend on thermodynamic conditions of the reaction (e.g., temperature, pressure, etc.). As another example, the reaction rate can depend on chemical conditions of the reaction (e.g., acidity, salinity, etc.).

The reaction rate for each chemical reaction within the pathway can also depend on certain catalysts and inhibitors mediating the reaction. For example, a given reaction can be facilitated by a catalyst (e.g., a catalyzing enzyme) for the reaction, and increasing a concentration of the catalyst can increase the rate of the reaction. Similarly, an inhibitor (e.g., an inhibitory metabolite) of a given reaction can hinder the reaction, and increasing a concentration of the inhibitor can decrease the rate of the reaction. The extent to which given concentrations of catalysts and inhibitors can mediate a given reaction can also depend on thermodynamic and chemical conditions (e.g., temperature, pressure, acidity, salinity, etc.) surrounding the reaction.

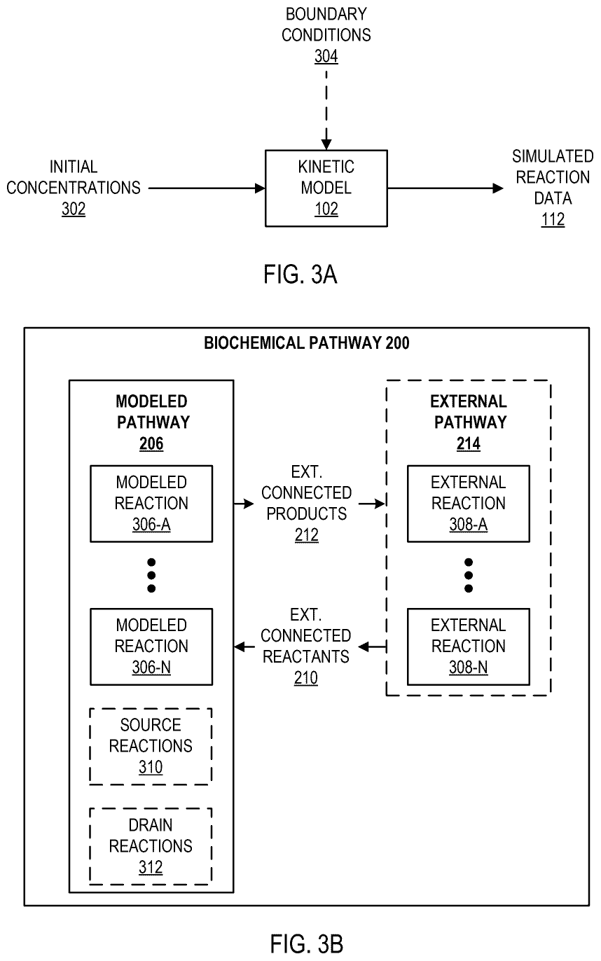

The kinetic model 102 can simulate the chemical reactions of the pathway to generate a variety of predictions for concentrations of the compounds in the pathway. As an example, the kinetic model 102 can process initial concentrations of the compounds and can simulate how the concentrations of the compounds change over time. As another example, the kinetic model 102 can simulate the chemical reactions of the pathway in order to predict steady-state concentrations of the compounds for equilibria of the pathway. In particular, the kinetic model 102 can include a variety of kinetic model parameters that determine how the model 102 simulates each chemical reaction in the pathway to change concentrations of compounds within the pathway over time. The kinetic model 102 is described in more detail below with reference to FIGS. 3A and 3B.

In general, the model generation system 100 can generate and train the kinetic model 102 to generate predicted concentrations of the compounds within the pathway.

The system 100 includes a model selection system 104 that can select a structure (e.g., an architecture, a functional form, etc.) for the kinetic model 102. In particular, the model selection system 104 can process pathway data 106 (e.g., data characterizing the chemical reactions within the pathway, such as stoichiometries, catalytic enzymes, inhibitors, enzymatic reaction mechanisms, reaction constants, etc.) to determine the structure for the kinetic model 102.

The system 100 includes a training system 108 that can generate updated model parameters 110 as part of training the kinetic model 102. In particular, the training system 108 can train the kinetic model 102 to generate simulated reaction data 112 (e.g., simulated data generated by the kinetic model 102 predicting compound concentrations resulting from the chemical reactions in the pathway) with a reduced discrepancy (e.g., error) with corresponding experimental reaction data 114 (e.g., experimentally observed compound concentrations resulting from the chemical reactions in the pathway).

The experimental reaction data 114 and the simulated reaction data 112 can include any of a variety of data specifying compound concentrations within the pathway. For example, the experimental reaction data 114 and the simulated reaction data 112 can include data characterizing concentrations of compounds within the biochemical pathway. As another example, the experimental reaction data 114 and the simulated reaction data 112 can include reaction flux data (e.g., data specifying rates of change of chemical concentrations) for chemical reactions in the biochemical pathway.

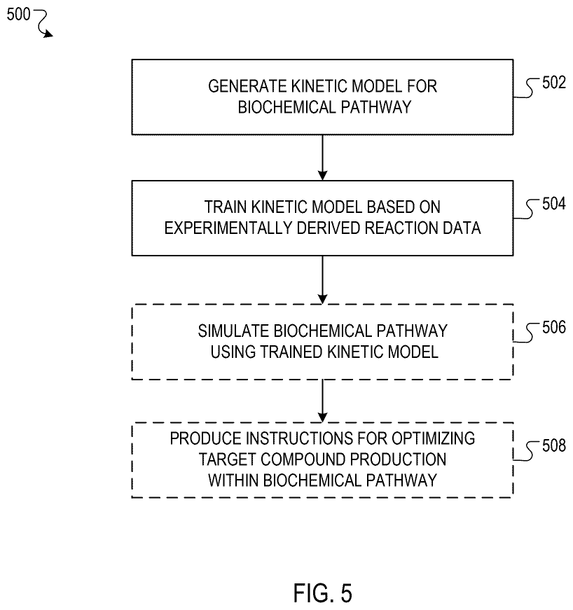

An example process for generating and training the kinetic model 102 to generate predicted concentrations of the compounds within the pathway is described in more detail below with reference to FIG. 5.

After training the kinetic model 102, the system 100 can use the model 102 for any of a variety of tasks regarding the biochemical pathway. For example, the task can be to increase a rate of production for a particular output (e.g., product chemical) of the biochemical pathway and system 100 can use the model 102 to predict the effect of a concentration of a target enzyme on the rate of production of the particular output of the biochemical pathway. As a further example, the system 100 can determine enzyme concentration parameters (e.g., by numerical optimization over a space of the enzyme concentration parameters) that optimize an objective function measuring the production rate of the particular output of the biochemical pathway. The system 100 can use the optimization results to identify a target enzyme.

The particular output (e.g., product chemical) of the biochemical pathway can be, e.g., a target pharmaceutical, biofuel, industrial, enzyme, and so on.

In some implementations, the system 100 can determine that a genome of a microorganism should be modified to increase expression (e.g., a rate of production) of the particular output of the biochemical pathway. For example, the system 100 can determine that certain gene sequences should be added to or removed from the genome of the microorganism (e.g., to increase expression of a catalyst within the biochemical pathway, to reduce an inhibitor concentration within the biochemical pathway, etc.) in order to increase expression of the particular output.

In some implementations, the system 100 can output instructions for genetically modifying the microorganism to increase the expression of the target enzyme. For example, the system 100 can identify a species or variant of the microorganism to use to produce the particular output and can output instructions for specific gene sequences to modify within the genome of the microorganism to increase expression of the particular output. As a further example, the system 100 can output instructions for modifying the specific gene sequences using, e.g., CRISPR-Cas9.

In some implementations, the system 100 can output instructions for cultivating a population of the genetically modified microorganisms. For example, the system 100 can determine particular nutrients and growth conditions for cultivating the genetically modified microorganisms. As a further example, the system 100 can determine nutrients and growth conditions for the genetically modified microorganisms that increase expression of the particular output from the biochemical pathway by the cultivated microorganisms.

In some implementations, the system can perform an optimization, over a space of bioreactor parameters, to optimize an output of the biochemical pathway as defined by the trained kinetic model. The space of bioreactor parameters can include, e.g., temperature parameters, pressure parameters, pH parameters, dissolved oxygen parameters, and so forth. The system can then transmit the optimized bioreactor parameters to a bioreactor and cause the bioreactor to implement experimental conditions defined by the optimized bioreactor parameters.

FIG. 2 illustrates an example full biochemical pathway 200 that includes chemical reactions 202-A through 202-E. Each of the reactions 202-A through 202-E is mediated by corresponding mediators (e.g., catalysts, inhibitors, etc.) 204-A through 204-E. As a particular example, the reaction 202-A is mediated by mediators 204-A, consumes reactants 203-A and products 208-E, and produces extrinsically connected reactants 210.

The system can generate and train a kinetic model to predict chemical concentrations for the pathway 200. The system can restrict the kinetic model to a subset of the reactions 202-A through 202-E by generating and training the model to predict chemical concentrations for a modeled pathway 206 that is a subset of the full biochemical pathway 200. For example, as illustrated in FIG. 2, the modeled pathway 206 includes the chemical reactions 202-B through 202-D.

The system can train the kinetic model to simulate the reactions 202-B, 202-C, and 202-D within the modeled pathway 206 and to predict concentrations of the products 208-B, 208-C, and 208-D over time as produced and consumed within the modeled pathway 206. The system can also train the kinetic model to simulate concentrations of extrinsically connected reactants 210 (e.g., compounds in the pathway 200 that are consumed within and produced outside the modeled pathway 206) and extrinsically connected products 212 (e.g., compounds in the pathway 200 that are produced within and consumed outside the modeled pathway 206) and to simulate how the extrinsically connected reactants 210 and products 212 influence the reactions 202-B through 202-D of the modeled pathway 206A.

In some implementations, the system can represent the chemical reactions 202-A and 202-E as an external pathway 214. In particular, the system can generate, train, and perform inference with the kinetic model by representing the extrinsically connected reactants 210 as being produced by using simplified source reactions for the modeled pathway 206 and by representing the extrinsically connected reactants 212 as being consumed by simplified drain reactions for the modeled pathway 206. By utilizing the simplified source and drain reactions, the system can train the kinetic model to accurately predict the concentrations of chemicals within the modeled pathway 206 without predicting concentrations for chemicals outside the modeled pathway. For example, as illustrated in FIG. 2, the system can train the kinetic model to predict chemical concentrations for the reactions 202-B through 202-D as part of the total pathway 200 without explicitly modeling the reactions 202-A and 202-E.

FIGS. 3A and 3B illustrate using a kinetic model 102 to simulate a biochemical pathway (e.g., the biochemical pathway 200 of FIG. 2).

FIG. 3A illustrates using the kinetic model 102 to generate the simulated reaction data 112 for the pathway based on initial concentrations 302. As described above, the simulated reaction data 112 can be, e.g., concentrations or rates of change of concentrations (e.g., fluxes) of the compounds (e.g., metabolites, enzymes, etc.) over a sequence of time steps, steady-state concentrations or rates of production (e.g., fluxes) for the compounds at equilibria of the pathway, etc. The initial concentrations 302 can be concentrations of compounds within the pathway at an initial time step.

Representing the concentrations of compounds within the pathway at a time t as a vector, \vec{x}ₜ, the kinetic model 102 can model rates of change of the concentrations of the compounds following:

$$\frac{d {\vec{x}}_{t}}{d t} = {f}_{θ} ( {\vec{x}}_{t} ; {\vec{c}}_{t} , {\vec{ι}}_{t} , {P}_{t} )$$

Where θ are kinetic model parameters of the model 102, \vec{c}ₜ represents concentrations of catalysts within the pathway at the time t, \vec{i}ₜ represents concentrations of inhibitors within the pathway at the time t, and Pₜ represents physical properties surrounding the pathway (e.g., thermodynamic properties, chemical properties, etc.).

The kinetic model 102 can predict the concentration rates of change by modeling linked reaction fluxes among the reactions of the pathway. For example, the kinetic model 102 can model rates of change of the concentrations for a reaction R following:

$$\frac{d {\vec{x}}_{t}^{R}}{d t} = {f}_{θ}^{R} ( {\vec{x}}_{t}^{R} ; {\vec{c}}_{t}^{R} , {\vec{ι}}_{t}^{R} , {P}_{t} )$$

Where

$${\vec{x}}_{t}^{R}$$

represents concentrations of compounds within the reaction R,

$${\vec{c}}_{t}^{R}$$

represents concentrations of catalysts within the reaction R, and

$${\vec{l}}_{t}^{R}$$

within the reaction R. As an example,

$$\frac{d {\vec{x}}_{t}^{R}}{dt}$$

can include a respective negative component for each reactant of the reaction R representing a rate at which the reactant is consumed by the reaction R and a respective positive component for each product of the reaction R representing a rate at which the product is produced by the reaction R.

When the kinetic model 102 models individual reaction fluxes for each of the reactions of the pathway, the model 102 can predict a reaction rate,

$${v}_{t}^{R} ,$$

for the reaction R following:

$${v}_{t}^{R} = {f}_{θ}^{R} ( {\vec{x}}_{t}^{R} ; {\vec{c}}_{t}^{R} , {\vec{l}}_{t}^{R} , {P}_{t} )$$

And can predict the reaction flux for the reaction R following:

$$\frac{d {\vec{x}}_{t}^{R}}{dt} = {v}_{t}^{R} {\vec{s}}^{R}$$

Where \vec{s}^{R} is a vector representing the stoichiometry of the reaction R (e.g., the relative rates at which compounds within the reaction are produced and consumed). For example, \vec{s}^{R} can include a respective negative component for each reactant of the reaction R representing a relative rate at which the reactant is consumed by the reaction R and a respective positive component for each product of the reaction R representing a relative rate at which the product is produced by the reaction R.

When the kinetic model 102 models individual reaction fluxes for each of the reactions of the pathway, the model 102 can predict the rates of change of concentration for compounds within the total pathway following:

$$\frac{d {\vec{x}}_{t}}{dt} = {\sum }_{R} \frac{d {\vec{x}}_{t}^{R}}{dt}$$

The kinetic model 102 can have any of a variety of architectures and functional forms to predict chemical concentrations within the pathway. As an example, the kinetic model 102 can be a neural network, and the kinetic model parameters, θ, can be parameters of the neural network. As another example, the kinetic model 102 can be a system of differential equations, and the kinetic model parameters, θ, can be parameters and coefficients for the system of differential equations.

The system can apply certain boundary conditions 304 to the kinetic model 102. In particular, a boundary condition 304 for a given compound within the pathway can require the kinetic model 102 to maintain, e.g., a fixed concentration of the given compound, a fixed rate of production or consumption of the compound, etc. while modeling the chemical reactions of the pathway. Throughout this specification, a boundary compound (e.g., a boundary metabolite) refers to a compound within the biochemical pathway to which the system has applied a boundary condition (e.g., a compound in the biochemical pathway that the system holds at a fixed concentration).

In some implementations, the kinetic model 102 can adhere to the boundary conditions 304 by modeling additional source or drain reactions (e.g., artificial source and drain reactions that may not themselves be present within the biochemical pathway in nature) for the compounds of the boundary conditions 304. The parameters of the additional source and drain reactions can be adjusted (e.g., by the system, by the kinetic model 102, etc.) to ensure that the kinetic model 102 follows the boundary conditions 304. As an example, the parameters of a source or a drain reaction for a given compound can be adjusted so that the kinetic model 102 maintains a fixed concentration of the given compound. As another example, the parameters of a source or a drain reaction for a given compound can be adjusted so that the kinetic model 102 produces or consumes the given compound at a fixed rate.

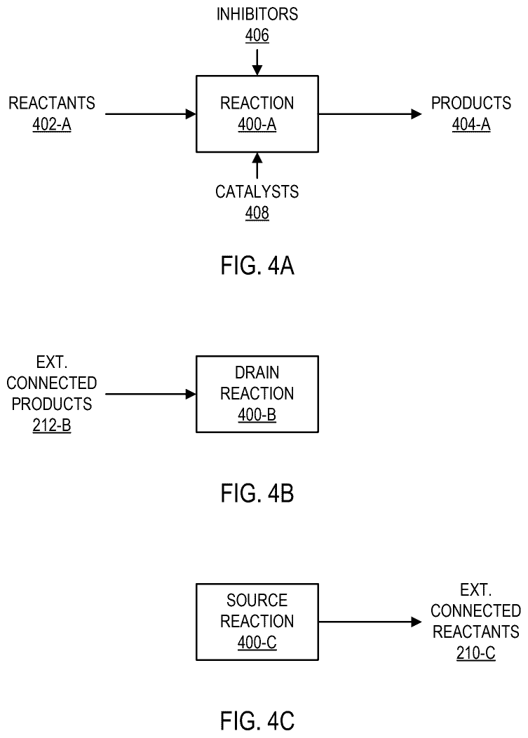

Examples of how the kinetic model 102 can model reactions from the pathway and model source and drain reactions to maintain the boundary conditions 304 are described in more detail below with reference to FIGS. 4A-4C.

FIG. 3B illustrates the biochemical pathway 200 as simulated by the kinetic model 102.

As discussed above, the kinetic model 102 can generate predictions for modeled reactions 306-A through 306-N, which form the modeled pathway 206 and can be a subset of the reactions within the complete pathway 200. The pathway 200 can include external reactions 308-A through 308-N (e.g., reactions that are external to the modeled pathway 206 that form the external pathway 216). Within the full pathway 200, extrinsically connected products 212 may exit the modeled pathway 206 and be consumed within the external pathway 214, and extrinsically connected reactants 210 may be produced within the external pathway 214 and enter the modeled pathway 206.

The modeled pathway 206 can include certain source reactions 310 and drain reactions 312 to model the extrinsically connected reactants 210 and products 212 entering and exiting the modeled pathway 206. When the model parameters for the source 310 and drain reactions 312 are selected (e.g., by the system, by the kinetic model 102, etc.) to replicate boundary conditions 304 within the complete pathway 200 for the extrinsically connected reactants 210 and products 212, the kinetic model 102 can generate predictions for modeled reactions 306-A through 306-N as a part of the complete pathway 200 while only simulating reactions within the modeled pathway 206.

FIGS. 4A-4C illustrate example chemical reactions simulated by a kinetic model (e.g., the kinetic model 102 of FIG. 1).

FIG. 4A illustrates an example chemical reaction 400-A of a biochemical pathway (e.g., one of the modeled reactions 306-A through 306-N of the pathway 200, as illustrated in FIG. 3B). As described above, the reaction 400-A consumes reactants 402 and generate products 404. The reaction 400-A can be mediated by inhibitors 406 and catalysts 408 for the reaction 400-A.

The kinetic model can predict changes to concentrations of the reactants 402 and products 404 resulting from the reaction 400-A. As described above, the kinetic model can predict rates of change for concentrations of the reactants 402 and products 404 due to the reaction 400-A based on concentrations of the reactants 402, products 404, inhibitors 406, and catalysts 408. As part of generating predictions for the reaction 400-A, the kinetic model can predict a reaction rate of the reaction 400-A based on the concentrations of the reactants 402, products 404, inhibitors 406, and catalysts 408.

For example, the kinetic model can predict the reaction rate of the reaction 400-A, denoted below as R, following:

$${v}^{R} = {f}_{R} ( \vec{x} , \vec{c} , \vec{l} ; {θ}_{R} )$$

Where v^{R} denotes the predicted reaction rate, \vec{x} denotes compound (e.g., reactants 402 and products 404) concentrations for the reaction 400-A, \vec{c} denotes catalyst 408 concentrations for the reaction 400-A, \vec{i} denotes inhibitor 406 concentrations for the reaction 400-A, and θ_{R} are reaction parameters for the reaction 400-A.

In some implementations, the system can retrieve data characterizing the reaction rate equation for the reaction 400-A (e.g., data characterizing the functional form of ƒ_{R}, the reaction parameters, θ_{R}, etc.). As an example, the system can retrieve reaction rate data from a database stored by the system. As another example, the system can retrieve reaction rate data from an external database, such as, e.g., the SABIO biochemical reaction rate database as described by Wittig et al. in “SABIO-RK—Database for Biochemical Reaction Kinetics”, the NIST chemical kinetics database as described by Mallard et al. in “NIST Chemical Kinetics Database”, and so on.

The system can determine the reaction parameters, θ_{R}, based on reaction constants for the reaction 400-A. The equilibrium constants for the reaction 400-A can include, e.g., equilibrium constants (K_{eq}), enzyme turnover rates (k_{cat}), dissociation constants (K_{d}), inhibition constants (Kᵢ), Michaelis-Menten constants (Kₘ), etc., for the reaction 400-A.

The kinetic model parameters can include the reaction parameters, θ_{R}, and the system can train the kinetic model by updating θ_{R}. In some implementations, the kinetic model parameters can include the reaction constants for the reaction 400-A that determine the reaction parameters, θ_{R}, and the system can train the kinetic model by updating the reaction constants for the reaction 400-A.

As described above, the kinetic model can simulate additional source and drain reactions to maintain boundary conditions for extrinsically connected reactants 210 and products 212 of the pathway.

FIG. 4B illustrates an example drain reaction 400-B for the extrinsically connected products 212-B. The kinetic model parameters for the source reaction 400-B can be adjusted (e.g., by the system, by the kinetic model, etc.) to maintain a particular outflux of the extrinsically connected products 212-B. As an example, the parameters of the 400-B source can be adjusted so that the kinetic model maintains fixed concentrations of the extrinsically connected products 212-B. As another example, the parameters of the 400-B source can be adjusted so that the kinetic model produces the extrinsically connected products 212-B at fixed rates.

FIG. 4C illustrates an example source reaction 400-C for the extrinsically connected reactants 210-C. The kinetic model parameters for the source reaction 400-C can be adjusted (e.g., by the system, by the kinetic model, etc.) to maintain a particular influx of the extrinsically connected reactants 210-C. As an example, the parameters of the 400-C source can be adjusted so that the kinetic model maintains fixed concentrations of the extrinsically connected reactants 210-C. As another example, the parameters of the 400-C source can be adjusted so that the kinetic model consumes the extrinsically connected reactants 210-C at fixed rates.

FIG. 5 is a flow diagram of an example process for generating and training a kinetic model (e.g., the kinetic model 102 of FIG. 1) for a biochemical pathway (e.g., the pathway 200 of FIG. 2). For convenience, the process 500 will be described as being performed by a system of one or more computers located in one or more locations. For example, a training system, e.g., the training system 100 of FIG. 1, appropriately programmed in accordance with this specification, can perform the process 500.

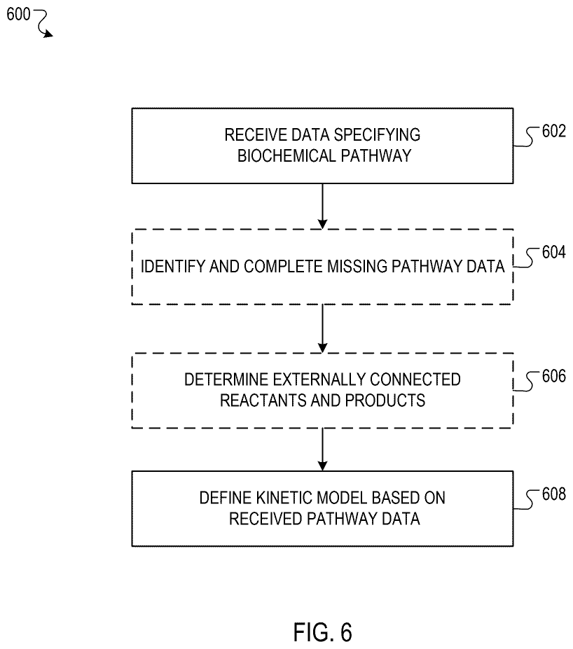

The system can generate the kinetic model for simulating the biochemical pathway (step 502). As described above, the kinetic model simulates how chemical reactions within the biochemical pathway change concentrations of compounds within the pathway over time. For example, the kinetic model can specify reaction rate equations for each of the simulated chemical reactions and the system can generate the kinetic model by determining, e.g., functional forms, initial parameter values, etc., for the reaction rate equations. An example process for generating the kinetic model is described in more detail below with reference to FIG. 6.

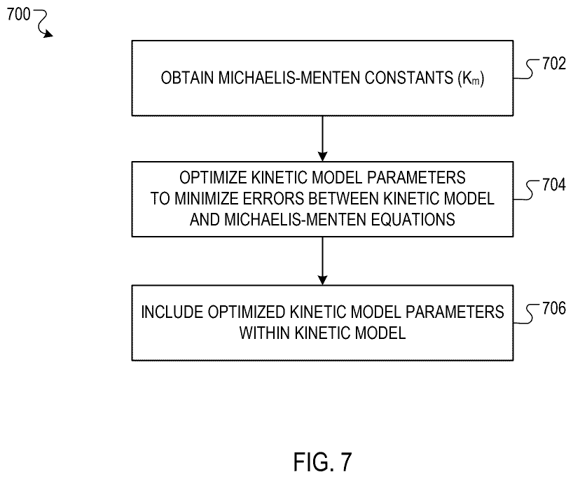

The system can train the kinetic model using experimentally derived data for the biochemical pathway (step 504). In particular, the system can train the kinetic model by updating parameters of the kinetic model to minimize errors for compound concentrations, reaction fluxes, etc., between simulations from the kinetic model and the experimentally derived data. An example process for training the kinetic model is described in more detail below with reference to FIG. 7.

In some implementations, the system can train the kinetic model parameters multiple times, each time with a different random initialization of values of the set of kinetic model parameters, to generate an ensemble of trained values of the set of kinetic model parameters.

The system can then use the trained kinetic model to simulate the biochemical pathway (step 506). In some implementations, the system can use the kinetic model to simulate the biochemical pathway in order to optimize production of target compounds with the biochemical pathway.

In particular, the kinetic model can process data characterizing concentrations of compounds within the biochemical pathway to predict rates of production and consumption of the compounds. For example, as described above, the kinetic model can simulate the biochemical pathway as a system of differential equations that includes a differential equation for each chemical reaction in the pathway that specifies rates of production (resp. consumption) of products (resp. reactants) of the chemical reaction based on concentrations of the reactants, products, catalysts, and inhibitors of the chemical reaction. The system can simulate the production and consumption of compounds within the biochemical pathway by analytically or numerically (e.g., using Euler's method, the Runge-Kutta methods, etc.) solving the system of differential equations for the pathway.

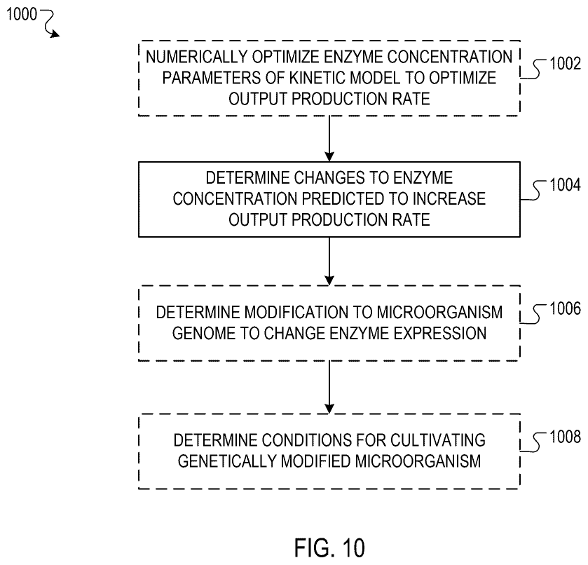

An example process for optimizing production of a target compound using the kinetic model is described in more detail below with reference to FIG. 10.

When the system generates an ensemble of trained values for the set of kinetic model parameters, the system can simulate the biochemical pathway with each of the trained kinetic model parameters to generate an ensemble of simulation results. The system can then optimize the production of the target compound based on the generated ensemble of simulation results. For example, the system can determine an aggregated result (e.g., an average) from the ensemble of simulation results and optimize the production of the target compound using the aggregated simulation result.

In some implementations, the system can produce instructions for optimizing the production of target compounds using the biochemical pathway (step 508).

FIG. 6 is a flow diagram of an example process for generating a kinetic model (e.g., the kinetic model 102 of FIG. 1) to simulate a biochemical pathway (e.g., the pathway 200 of FIG. 2). For convenience, the process 600 will be described as being performed by a system of one or more computers located in one or more locations. For example, a training system, e.g., the training system 100 of FIG. 1, appropriately programmed in accordance with this specification, can perform the process 600.

The system can receive pathway data specifying the biochemical pathway (step 602). In general, the pathway data characterizes chemical reactions in within the biochemical pathway.

The system can receive the pathway data from any of a variety of sources. For example, the system can receive the pathway data as provided by a user of the system. As another example, the system can receive the pathway data as generated by an external system.

The system can determine any of a variety of properties for the chemical reactions characterized by the pathway data, e.g., stoichiometries, catalytic enzymes, inhibitors, enzymatic reaction mechanisms, reaction constants, etc., for the reactions of the pathway. For example, the system can determine, e.g., equilibrium constants (K_{eq}), enzyme turnover rates (k_{cat}), dissociation constants (K_{d}), inhibition constants (Kᵢ), Michaelis-Menten constants (Kₘ), etc., for the reactions of the pathway.

As an example, the pathway data can include one or more properties for the chemical reactions of the pathway. As another example, the system can determine one or more properties for the chemical reactions by retrieving data from databases storing chemical reaction data (e.g., external chemical reaction databases, chemical reaction databases stored by the system, etc.).

The pathway data can specify a subset (e.g., a modeled pathway) of the chemical reactions within the biochemical pathway for the kinetic model to predict. For example, the modeled pathway can be a subset of a genome scale model (e.g., a model of a complete metabolism of an organism) as limited to chemical reactions related to producing a target output compound.

The system can process the received pathway data to automatically generate data defining the kinetic model for the biochemical pathway following steps 604 through 608, as described below.

In some implementations, the system can process the pathway data to identify chemical reactions with incomplete chemical reaction data (step 604). When the system identifies a chemical reaction as having incomplete chemical reaction data, the system can automatically complete the missing data for the chemical reaction. In particular, the system can identify features (e.g., stoichiometries, catalyzing enzymes, inhibitors, enzyme reaction mechanisms, etc., of reactions in the pathway) that are missing from the pathway data and can retrieve the missing features. For example, when the system determines that the pathway data is missing a feature for a reaction in the biochemical pathway, the system can automatically retrieve the missing feature for the reaction from a database (e.g., from a database of chemical reaction data).

In particular, for each of the chemical reactions with incomplete chemical reaction data, the system can generate, based on the pathway data, a query characterizing the chemical reaction (e.g., based on the stoichiometry of the reaction, identities of the reactants, products, catalysts, and inhibitors of the chemical reaction, etc.). The system can use an application programming interface (API) for the database to process the queries and retrieve values for the missing features as stored within the database. As an example, for each chemical reaction with incomplete chemical reaction data, the system can use the API to process the query, identify a unique corresponding database entry for the chemical reaction within the database, and retrieve the values for the missing features as specified by the database entry for the chemical reaction.

In some implementations, the system can determine extrinsically connected reactants and products within the pathway (step 606). In general, the extrinsically connected reactants and products are produced or consumed within the modeled pathway that can be produced or consumed outside of the modeled pathway.

The system can identify a variety of compounds within the modeled pathway as boundary compounds. For example, the system can identify compounds that are consumed or produced in only one chemical reaction of the modeled pathway as being boundary compounds (e.g., boundary metabolites). As another example, the system can identify reactants or products of an irreversible chemical reactions in the modeled pathway as being boundary compounds. The system can generate the kinetic model to hold concentrations of the boundary compounds constant (e.g., rather than having variable values).

The system can identify a variety of compounds within the modeled pathway as extrinsically connected reactants or products. For example, the system can identify compounds in the modeled pathway that are also included in chemical reactions outside the modeled pathway (e.g., chemical reactions from the complete biochemical pathway, from a genome scale model of a biological process including the modeled pathway etc.). As another example, when the system identifies a compound as a boundary compound, the system can further identify the compound as an extrinsically connected reactant or as an extrinsically connected product.

The system can include additional source and drain reactions within the kinetic model that simulate the entry of the extrinsically connected reactants from external sources and the exit of the extrinsically connected products from the modeled pathway. The kinetic model can simulate the additional source and drain reactions by any appropriate method. For example, the kinetic model can fix the concentration of an extrinsically connected reactant or product to a particular value. As another example, the kinetic model can fix the influx rate (resp. outflux rate) of an extrinsically connected reactant (resp. product) to a particular value. As another example, the kinetic model can include kinetic model parameters for the source and drain reactions and can additionally train the kinetic model to replicate experimental data for the extrinsically connected reactants and products.

For example, the system can determine expected fluxes of the extrinsically connected reactants (resp. products) into (resp. out from) the modeled pathway and can train the kinetic model to replicate the expected fluxes. As an example, the system can determine the expected fluxes based on experimental data (e.g., experimental data for the biochemical pathway, for a genome-scale model of a process including the modeled pathway, etc.). As another example, the system can determine (e.g., by numerical optimization) production and consumption rates of the extrinsically connected reactants and products in reactions from a process that includes the modeled pathway (e.g., the biochemical pathway, a genome-scale model of a process including the modeled pathway, etc.). The system can determine the expected fluxes by adding together the determined production and consumption rates for reactions outside the modeled pathway.

The system can define the kinetic model based on the pathway data (step 608). The kinetic model can include a variety of kinetic model parameters that determine how the kinetic model simulates the reactions within the modeled pathway. In particular, the kinetic model can include kinetic model parameters (e.g., equilibrium constants (K_{eq}), enzyme turnover rates (k_{cat}), dissociation constants (K_{d}), inhibition constants (Kᵢ), drain reaction constants (K_{drain}), enzyme concentrations, etc.) for each of the modeled reactions.

In some implementations, the system can identify respective reaction rate expressions for each of the modeled reactions parametrized at least in part by kinetic model parameters of the kinetic model. The system can identify the reaction rate expressions for the modeled reactions (e.g., by querying a chemical reaction database) based on, e.g., numbers of reactants and products, identities of reactants and products, enzymatic reaction mechanisms, etc., of the reactions. The system can process the identified reaction rate expressions to define the kinetic model of the biological pathway. In particular, as part of defining the kinetic model, the system can generate a model of a rate of change of a concentration of each compound in the modeled pathway with respect to time as a combination of the reaction rate expressions for each chemical reaction that includes the compound in the biochemical pathway. As an example, for each compound within the modeled pathway, the system can model the rate of change of the concentration of the compound by summing respective rates of change of the compound caused by each reaction in the pathway that includes the compound.

As part of defining the kinetic model, the system can initialize values for the kinetic model parameters by a variety of methods. For example, the system can automatically retrieve initial values for some or all of the kinetic model parameters from databases of chemical reaction data.

As a further example, for each kinetic model parameter, the system can generate a query characterizing the kinetic model parameter (e.g., specifying a particular enzyme, a particular chemical reaction, a particular kinetic model parameter type, etc.). The system can use APIs for the databases to process the queries and retrieve initial values for the kinetic model parameters as stored within the database. As an example, for each kinetic model parameter, the system can use the APIs to process the query, identify a unique corresponding database entry for the kinetic model parameter from the databases, select one of the database entries for the kinetic model parameter, and retrieve the initial value for the kinetic model parameter as specified by the selected database entry for the kinetic model parameter.

As another example, the system can train (e.g., pre-train) the kinetic model using experimental data that characterizes the modeled reactions in isolation (e.g., that characterizes reactant and product concentrations for the modeled reactions progressing without external influences). An example process for pre-training the kinetic model using the experimental data that characterizes the modeled reactions in isolation is described in more detail below with respect to FIG. 7.

When the modeled pathway includes extrinsically connected reactants and products, the kinetic model parameters can include boundary metabolite concentration values for the extrinsically connected reactants and products.

In some implementations, the kinetic model parameters can include (i) parameters identified as global kinetic model parameters that are invariant across experimental conditions for the modeled pathway and (ii) parameters that are identified as local kinetic model parameters that vary across experimental conditions for the modeled pathway. As an example, the global kinetic model parameters can include enzymatic parameters (e.g., enzyme turnover rates (k_{cat}), dissociation constants (K_{d}), or inhibition constants (Kᵢ), etc.). As another example, the local kinetic model parameters can include boundary metabolite concentration values for the extrinsically connected reactants and products.

FIG. 7 is a flow diagram of pre-training the kinetic model using experimental data that characterizes modeled chemical reactions in isolation. For convenience, the process 700 will be described as being performed by a system of one or more computers located in one or more locations. For example, a training system, e.g., the training system 100 of FIG. 1, appropriately programmed in accordance with this specification, can perform the process 700.

The system can obtain Michaelis-Menten constants (Kₘ) associated with certain enzymes in the modeled pathway (step 702). Each Michaelis-Menten constant characterizes properties of an associated chemical reaction in isolation of any other chemical reactions.

The system can generate chemical reaction flux values for modeled reactions that include the certain enzymes using Michaelis-Menten equations parametrized by the obtained Michaelis-Menten constants (Kₘ).

The system can train some or all of the kinetic model parameters (e.g., dissociation constants (K_{d}) for enzymes within the modeled pathway) by optimizing an error between the chemical reaction flux values generated by the Michaelis-Menten equations and chemical reaction flux values generated using the kinetic model (step 704).

For example, for a reaction flux value v over a sequence of time steps, the optimized error can be an L2 loss, following:

$$L = {\sum }_{t} {\vert {v}_{t}^{*} - {v}_{t} \vert}^{2}$$

Where

$${v}_{t}^{*}$$

is the reaction flux value at time t generated by the Michaelis-Menten equations and vₜ is the reaction flux value at time t generated by the kinetic model.

The system can finally include the trained kinetic model parameters (e.g., optimized dissociation constants (K_{d}) within the kinetic model (step 706).

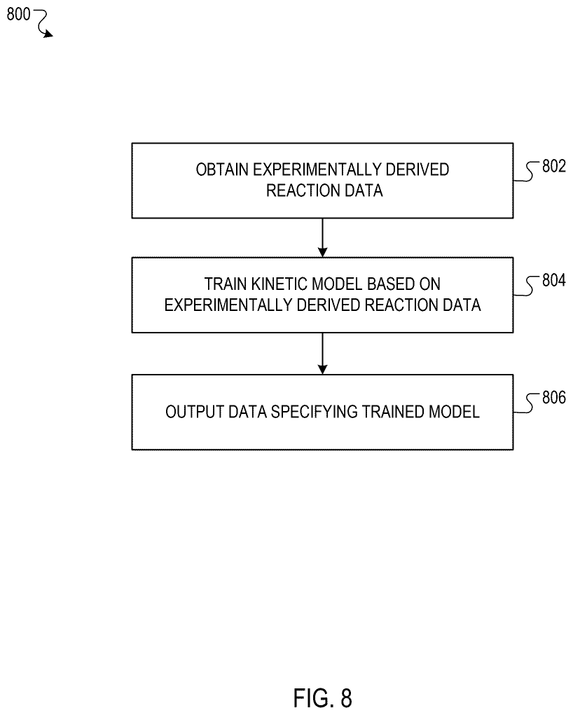

FIG. 8 is a flow diagram of an example process for training a kinetic model (e.g., the kinetic model 102 of FIG. 1) to simulate a biochemical pathway (e.g., the pathway 200 of FIG. 2). For convenience, the process 800 will be described as being performed by a system of one or more computers located in one or more locations. For example, a training system, e.g., the training system 100 of FIG. 1, appropriately programmed in accordance with this specification, can perform the process 800.

The system can obtain experimental reaction data that characterizes the modeled pathway (step 802). In particular, the experimental reaction data can characterize concentrations of compounds within the modeled pathway. As an example, the experimental reaction data can include measurements of concentrations of the reactants and products of the modeled reactions. As another example, the experimental reaction data can include reaction flux data for the modeled reactions.

In some implementations, the system can obtain experimental reaction data for multiple experimental conditions.

The system can train the kinetic model using the experimental reaction data (step 804).

In particular, the system can train the kinetic model using the experimental reaction data by optimizing (e.g., using a numerical optimization technique) an objective function that measures a discrepancy between: (i) simulated data generated by the kinetic model, and (ii) the experimental reaction data.

As an example, for a given compound within the modeled pathway, the objective function can measure an L2 loss based on concentrations of the given compound over a sequence of time steps, following:

$$ℒ = {\sum }_{t} {\vert {x}_{t}^{*} - {x}_{t} \vert}^{2}$$

Where

$${x}_{t}^{*}$$

is a concentration of the given compound within the modeled pathway at time t from the experimental reaction data and xₜ is a concentration of the given compound within the modeled pathway at time t as generated by the kinetic model.

As another example, for a given compound within the modeled pathway, the objective function can measure an L2 loss based on concentrations of the given compound over a sequence of time steps, following:

$$ℒ = {\sum }_{t} {\vert {x}_{t}^{*} - {\dot{x}}_{t} \vert}^{2}$$

Where

$${\dot{x}}_{t}^{*}$$

is a rate of change of the concentration of the given compound within the modeled pathway at time t from the experimental reaction data and \dot{x}ₜ is a rate of change of the concentration of the given compound within the modeled pathway at time t as generated by the kinetic model.

When the modeled pathway includes extrinsically connected reactants and products, the objective function can measure a discrepancy between: (i) simulated fluxes for the additional source and drain reactions generated using the kinetic model, and (ii) the expected fluxes for the additional source and drain reactions (as determined in step 804).

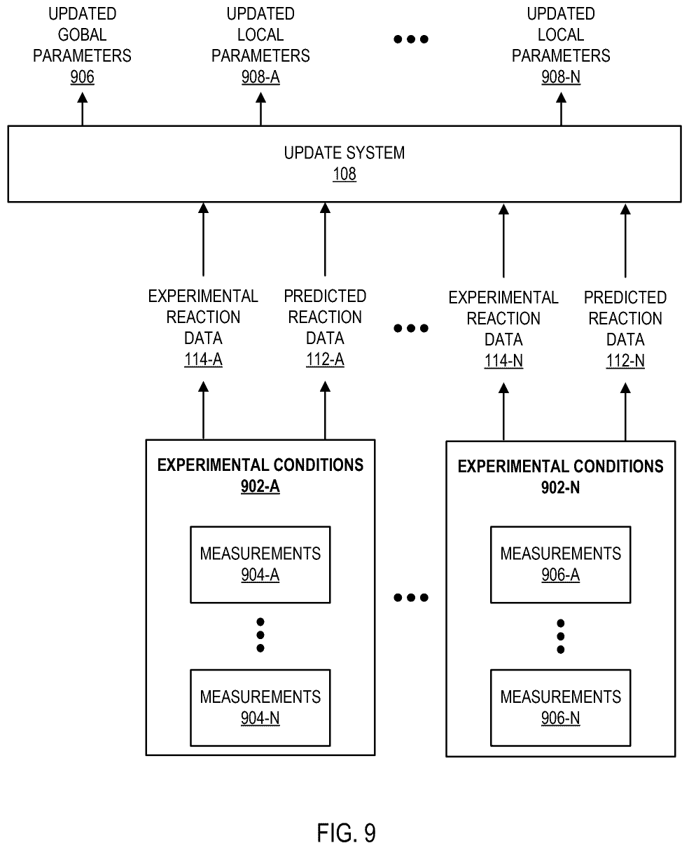

When the experimental reaction data includes data from multiple experimental conditions, the system can update the global kinetic model parameters based on the reaction data all of the experimental conditions and can update the local kinetic model parameters based only the reaction data for experimental conditions corresponding to the local kinetic model parameters. Updating the kinetic model parameters based on multiple experimental conditions is described in more detail below with reference to FIG. 9.

The system can finally output the trained kinetic model (step 806).

FIG. 9 illustrates using a training system to train a kinetic model using experimental data from different experimental conditions.

As illustrated in FIG. 9, the training system 102 can train the kinetic model using experimental data from the experimental conditions 902-A through 902-N. In particular, the experimental data includes data collected for measurements of the concentrations of compounds within the modeled pathway under each of the experimental conditions 902-A through 902-N. For example, the measurements 904-A through 904-N are collected under the experimental conditions 902-A and the measurements 906-A through 906-N are collected under the experimental conditions 902-N.

As described above, the kinetic model can include global kinetic parameters and local kinetic parameters. The global kinetic model parameters (e.g., enzymatic parameters) are invariant across different experimental conditions. The local kinetic model parameters (e.g., boundary conditions for extrinsically connected products and reactants) can vary across different experimental conditions.

Following the methods described above, the training system 102 trains the kinetic model parameters of the kinetic model by comparing experimental reaction data 114-A through 114-N with corresponding simulated reaction data 112-A through 112-N from the kinetic model. The reaction data 114-A through 114-N and the simulated reaction data 112-A through 112-N are generated under corresponding experimental conditions 902-A through 902-N.

As part of training the kinetic model, the training system 108 can determine updates for the global kinetic model parameters 906 based on all of the experimental reaction data 114-A through 114-N and simulated reaction data 112-A through 112-N.

The local kinetic model parameters 908-A through 908-N can be specific to the corresponding experimental conditions 902-A through 902-N. For example, the appropriate boundary conditions for extrinsically connected products and reactants may change between different experimental conditions 902-A through 902-N. Therefore, the training system 108 can determine updates for the local kinetic model parameters 908-A through 908-N based only on the corresponding experimental reaction data 114-A through 114-N and simulated reaction data 112-A through 112-N.

FIG. 10 is a flow diagram of an example process for using a kinetic model (e.g., the kinetic model 102 of FIG. 1) to optimize production of a target compound within a biochemical pathway (e.g., the pathway 200 of FIG. 2). For convenience, the process 1000 will be described as being performed by a system of one or more computers located in one or more locations. For example, a training system, e.g., the training system 100 of FIG. 1, appropriately programmed in accordance with this specification, can perform the process 1000.

In general, the system can use the kinetic model to determine changes to enzyme concentrations in the biochemical pathway predicted to increase the output production rate of the target compound. For example, the system can determine that increasing a concentration of a target enzyme is predicted to increase a rate of production of an output of the biochemical pathway.

The target compound may be, e.g., a pharmaceutical, a biofuel, an industrial enzyme, and so on.

In some implementations, the system can numerically optimize enzyme concentration parameters of the kinetic model to maximize a predicted production of the target compound within the biochemical pathway (step 1002). In particular, the system can perform a numerical optimization of an objective function that measures a production rate of the target compound (e.g., as simulated by the kinetic model) produced by the biochemical pathway over a space of possible values of the enzyme concentration parameters. The system can use any of a variety of optimization methods to numerically optimize the objective function for the production rate of the target compound. For example, the system may compute a gradient of the objective function with respect to the enzyme concentration parameters and may optimize the objective function by performing a gradient optimization technique (e.g., gradient descent, conjugate gradient descent, etc.). As another example, the system can optimize the objective function by performing a black-box optimization technique (e.g., an optimization technique that does not rely on computing gradients of the objective function), such as grid search, simulated annealing, evolutionary optimization, and so on.

As a further example, the system can perform multiple numerical optimizations of the objective function for the production rate of the target compound produced by the biochemical pathway. Each of the multiple numerical optimizations can optimize the production rate of the target compound with respect to a particular enzyme concentration parameter for the optimization while holding the other enzyme concentration parameters at a constant value.

The system can determine changes to enzyme concentrations in the biochemical pathway predicted to increase the output production rate of the target compound (step 1004). For example, the system can identify one or more target enzymes predicted to increase the output production rate of the target compound based on a result of the numerical optimization of the objective function for the production rate of the target compound. As a further example, when the system performs multiple numerical optimizations, each for a different enzyme concentration parameter, the system can identify the enzyme of the numerical optimization with the largest optimized output production rate as the target enzyme.

In some implementations, the system can determine that a genome of a microorganism should be modified in order to change the expression of the target enzyme (step 1006). In some implementations, the system can output instructions for genetically modifying the microorganism to increase the expression of the target enzyme. For example, the system can determine a genetic modification to the microorganism to increase the expression of a catalyzing enzyme for the target compound and can produce instructions for performing the determined genetic modification (e.g., using CRISPR-CaS9). For example, the genetic modification can include inserting a gene sequence for the catalyzing enzyme into the genome of the microorganism. As another example, the genetic modification can include inserting gene sequences for promoters of the expression of the catalyzing enzyme (e.g., promoters for translation, transcription, etc.) into the genome of the microorganism. As another example, the genetic modification can include removing gene sequences for repressors of the expression of the catalyzing enzyme (e.g., repressors for translation, transcription, etc.).

In some implementations, the system can determine and output instructions (e.g., optimized nutrients, growth conditions, etc.) for cultivating a population of the genetically modified microorganisms (step 1008).

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
receiving data characterizing a plurality of chemical reactions included in a biochemical pathway;
processing the data characterizing the plurality of chemical reactions included in the biochemical pathway to automatically generate data defining a kinetic model of the biochemical pathway, wherein the kinetic model comprises a set of kinetic model parameters;
modifying the kinetic model to apply one or more boundary conditions of the biochemical pathway to account for effects of chemical reactions outside the biochemical pathway on reactions included in the biochemical pathway;
obtaining experimental data characterizing the biochemical pathway, including one or both of:
metabolite concentration data measuring concentrations of one or more metabolites included in one or more chemical reactions in the biochemical pathway, and
reaction flux data for one or more chemical reactions included in the biochemical pathway;
training the set of kinetic model parameters of the kinetic model on the experimental data characterizing the biochemical pathway using a numerical optimization technique to optimize an objective function that measures a discrepancy between: (i) simulated data characterizing the biochemical pathway that is generated using the kinetic model, and (ii) the experimental data characterizing the biochemical pathway; and
outputting the kinetic model of the biochemical pathway after training the set of kinetic model parameters.

2. The method of claim 1, wherein obtaining the experimental data characterizing the biochemical pathway comprises:
obtaining respective experimental data characterizing the biochemical pathway under each of a plurality of respective experimental conditions.

3. The method of claim 2, wherein the set of kinetic model parameters comprises: (i) one or more kinetic model parameters identified as global kinetic model parameters that are invariant across experimental conditions, and (ii) one or more kinetic model parameters that are identified as local kinetic model parameters that vary across experimental conditions; and
wherein training the set of kinetic model parameters on the experimental data characterizing the biochemical pathway comprises:
determining a respective value of each global kinetic model parameter by training the global kinetic model parameters on experimental data corresponding to each of the plurality of experimental conditions; and
determining, for each experimental condition of the plurality of experimental conditions, a respective value of each local kinetic model parameter that is specific to the experimental condition by training the local kinetic model parameters only on experimental data corresponding to the experimental condition.

4. The method of claim 3, wherein the global kinetic model parameters comprise enzymatic parameters including one or more of: one or more enzyme turnover rates (k_{cat}), one or more dissociation constants (K_d), or one or more inhibition constants (K_i).

5. The method of claim 3, wherein the local kinetic model parameters comprise one or more boundary metabolite concentrations.

6. The method of claim 1, wherein training the set of kinetic model parameters of the kinetic model on the experimental data characterizing the biochemical pathway comprises:
performing the training of the set of kinetic model parameters a plurality of times, each time with a different random initialization of values of the set of kinetic model parameters, to generate an ensemble of trained values of the set of kinetic model parameters.

7. The method of claim 1, wherein processing the data characterizing the plurality of chemical reactions included in the biochemical pathway to automatically generate data defining a kinetic model of the biochemical pathway comprises:
automatically identifying a respective reaction rate expression for each of the plurality of chemical reactions, wherein each reaction rate expression is parametrized by one or more respective kinetic model parameters of the kinetic model; and
processing the reaction rate expressions for the plurality of chemical reactions to generate the data defining the kinetic model of the biochemical pathway.

8. The method of claim 7, wherein the biochemical pathway comprises a plurality of metabolites, wherein each metabolite is included in one or more chemical reactions in the biochemical pathway as a reactant or as a product; and
wherein processing the reaction rate expressions for the plurality of chemical reactions to generate the data defining the kinetic model of the biochemical pathway comprises, for each of one or more metabolites included in the biochemical pathway:
generating a model of a rate of change of a concentration of the metabolite with respect to time as a combination of the reaction rate expressions for each chemical reaction that includes the metabolite in the biochemical pathway.

9. The method of claim 7, wherein for one or more of the plurality of chemical reactions, automatically identifying a respective reaction rate expression for the chemical reaction comprises:
automatically identifying the reaction rate expression for the chemical reaction based on one or more of: a number of reactants in the chemical reaction; a number of products of the chemical reaction; or an enzymatic reaction mechanism of the chemical reaction.

10. The method claim 1, wherein modifying the kinetic model to apply one or more boundary conditions of the biochemical pathway to account for effects of chemical reactions outside the biochemical pathway on reactions included in the biochemical pathway comprises:
identifying, as a boundary metabolite, each metabolite in the biochemical pathway that is: included in only one chemical reaction in the biochemical pathway, or is included only as a reactant or only as a product of an irreversible chemical reaction in the biochemical pathway, or both; and
modifying the kinetic model to set, for each metabolite identified as a boundary metabolite, a concentration of the metabolite to be a constant instead of a variable value.

11. The method claim 1, wherein modifying the kinetic model to apply one or more boundary conditions of the biochemical pathway to account for effects of chemical reactions outside the biochemical pathway on reactions included in the biochemical pathway comprises:
identifying, as an extrinsically-connected metabolite, each metabolite in the biochemical pathway that is included in one or more chemical reactions outside the biochemical pathway in a genome-scale model of metabolism; and
modifying the kinetic model to include, for each extrinsically-connected metabolite, a respective drain chemical reaction that consumes the extrinsically-connected metabolite.

12. The method of claim 11, wherein modifying the kinetic model to apply one or more boundary conditions of the biochemical pathway to account for effects of chemical reactions outside the biochemical pathway on reactions included in the biochemical pathway further comprises:
determining, for each of one or more drain chemical reactions, a respective expected flux of the drain chemical reaction using the genome-scale model of metabolism; and
wherein, for each of one or more drain chemical reactions, the objective function used for training the set of kinetic model parameters of the kinetic model further measures a discrepancy between: (i) a simulated flux of the drain chemical reaction that is generated using the kinetic model, and (ii) the expected flux of the drain chemical reaction.

13. The method of claim 12, wherein determining, for each of one or more drain chemical reactions, the respective expected flux of the drain chemical reaction comprises:
obtaining experimental data characterizing respective uptake or production rates of one or more metabolites;
determining, based on the experimental data characterizing the respective uptake or production rates of the one or more metabolites and using a numerical optimization, a respective flux of each chemical reaction in the genome-scale model of metabolism; and
determining, for each drain chemical reaction associated with a metabolite, the respective expected flux as a combination of fluxes of chemical reactions in the genome-scale model of metabolism that: (i) produce or consume the metabolite, and (ii) are not included in the biochemical pathway.

14. The method of claim 1, wherein the set of kinetic model parameters of the kinetic model of the biochemical pathway comprise one or more of:
one or more equilibrium constants (K_{eq}); or
one or more enzyme turnover rates (k_{cat}); or
one or more dissociation constants (K_d); or
one or more inhibition constants (K_i); or
one or more drain reaction constants (K_{drain}); or
one or more enzyme concentrations; or
one or more boundary metabolite concentrations.

15. The method claim 1, wherein processing the data characterizing the plurality of chemical reactions included in the biochemical pathway to automatically generate data defining the kinetic model of the biochemical pathway comprises, for one or more kinetic model parameters of the kinetic model:
automatically retrieving data specifying a respective initial value of the kinetic model parameter from one or more databases of chemical reaction data.

16. The method claim 1, wherein processing the data characterizing the plurality of chemical reactions included in the biochemical pathway to automatically generate data defining the kinetic model of the biochemical pathway comprises:
obtaining one or more Michaelis-Menten constants (K_m) associated with an enzyme;
determining one or more dissociation constants (K_d) for the enzyme from the one or more Michaelis-Menten constants (K_m) associated with the enzyme, comprising:
performing a numerical optimization to determine optimized values of the one or more dissociation constants (K_d) that minimize an error between: (i) predicted chemical reaction flux values generated using a Michaelis-Menten equation parametrized by the one or more Michaelis-Menten constants (K_m), and (ii) predicted chemical reaction flux values generated using a kinetic model parametrized by the one or more dissociation constants (K_d); and
after optimizing values of the one or more dissociation constants (K_d), including the one or more dissociation constants (K_d) in the set of kinetic model parameters.

17. The method of claim 1, wherein processing the data characterizing the plurality of chemical reactions included in the biochemical pathway to automatically generate data defining the kinetic model of the biochemical pathway comprises:
processing the data characterizing the plurality of chemical reactions to identify one or more chemical reactions with incomplete chemical reaction data; and
automatically completing the chemical reaction data for each chemical reaction that is identified as having incomplete chemical reaction data, comprising, for each chemical reaction that is identified as having incomplete chemical reaction data:
automatically identifying one or more features that are not included in the received data characterizing the chemical reaction; and
automatically retrieving data specifying the one or more features that are not included in the received data characterizing the chemical reaction from one or more databases of chemical reaction data.

18. The method of claim 17, wherein for one or more of the chemical reactions that are identified as having incomplete reaction data, automatically retrieving data specifying the one or more features that are not included in the received data characterizing the chemical reaction from the database of chemical reaction data comprises automatically retrieving data specifying one or more of:
a stoichiometry of the chemical reaction; or
one or more catalyzing enzymes for the chemical reaction; or
one or more inhibitor metabolites for the chemical reaction; or
an enzymatic reaction mechanism for the chemical reaction.

19. A system comprising:
one or more computers; and
one or more storage devices communicatively coupled to the one or more computers, wherein the one or more storage devices store instructions that, when executed by the one or more computers, cause the one or more computers to perform operations comprising:
receiving data characterizing a plurality of chemical reactions included in a biochemical pathway;
processing the data characterizing the plurality of chemical reactions included in the biochemical pathway to automatically generate data defining a kinetic model of the biochemical pathway, wherein the kinetic model comprises a set of kinetic model parameters;
modifying the kinetic model to apply one or more boundary conditions of the biochemical pathway to account for effects of chemical reactions outside the biochemical pathway on reactions included in the biochemical pathway;
obtaining experimental data characterizing the biochemical pathway, including one or both of:
metabolite concentration data measuring concentrations of one or more metabolites included in one or more chemical reactions in the biochemical pathway, and reaction flux data for one or more chemical reactions included in the biochemical pathway;
training the set of kinetic model parameters of the kinetic model on the experimental data characterizing the biochemical pathway using a numerical optimization technique to optimize an objective function that measures a discrepancy between: (i) simulated data characterizing the biochemical pathway that is generated using the kinetic model, and (ii) the experimental data characterizing the biochemical pathway; and
outputting the kinetic model of the biochemical pathway after training the set of kinetic model parameters.

20. One or more non-transitory computer storage media storing instructions that when executed by one or more computers cause the one or more computers to perform operations comprising:
receiving data characterizing a plurality of chemical reactions included in a biochemical pathway;
processing the data characterizing the plurality of chemical reactions included in the biochemical pathway to automatically generate data defining a kinetic model of the biochemical pathway, wherein the kinetic model comprises a set of kinetic model parameters;
modifying the kinetic model to apply one or more boundary conditions of the biochemical pathway to account for effects of chemical reactions outside the biochemical pathway on reactions included in the biochemical pathway;
obtaining experimental data characterizing the biochemical pathway, including one or both of:
metabolite concentration data measuring concentrations of one or more metabolites included in one or more chemical reactions in the biochemical pathway, and
reaction flux data for one or more chemical reactions included in the biochemical pathway;
training the set of kinetic model parameters of the kinetic model on the experimental data characterizing the biochemical pathway using a numerical optimization technique to optimize an objective function that measures a discrepancy between: (i) simulated data characterizing the biochemical pathway that is generated using the kinetic model, and (ii) the experimental data characterizing the biochemical pathway; and
outputting the kinetic model of the biochemical pathway after training the set of kinetic model parameters.
